(19) United States
(12) Patent Application Publication
(10) Pub. No.: US 2021/0345709 A1
(43) Pub. Date: Nov. 11, 2021

(54) DISINFECTION EQUIPMENT OF FACEMASKS THROUGH UV-C LIGHT RADIATION

(71) Applicant: ALDO IVAN NOVELO ASCENCIO, ZAPOPAN (MX)

(72) Inventor: ALDO IVAN NOVELO ASCENCIO, ZAPOPAN (MX)

(21) Appl. No.: 16/874,569

(22) Filed: May 14, 2020

(30) Foreign Application Priority Data
May 6, 2020 (MX) MX/E/2020/021781

(51) Int. Cl. A41D 13/11; A61L 2/10

(52) U.S. Cl. CPC A41D 13/1192; A61L 2202/14; A61L 2202/11; A61L 2/10

(57) ABSTRACT

This utility model applies germicidal ultra violet light from 220 nm to 290 nm, also known as UV-C light, to facemasks for their disinfection and safely reuse by users; such application of ultraviolet light is executed by a piece of equipment with UV-C LED lights strategically installed in the upper and bottom parts of such equipment.

### DETAILED DESCRIPTION OF THE INVENTION

This utility model consists of an equipment that radiates UV-C light to facemasks for proper disinfection and thus their reuse. The equipment has 4 upper UV-C LED lights (1) and 2 bottom UV-C LED lights (2), strategically placed, that generate UV-C light to completely disinfect the target.

The equipment uses 4 upper UV-C LED lights (1) and 2 bottom UV-C LED lights (2) that radiate UV-C light, strategically placed to radiate UV-C light in both the upper and bottom parts of the facemask, such UV-C LED lights (1 and 2) have an upper cooling system (3) and a bottom cooling system (4) that prevents and avoids overheating of the lights and allows the continuous use of the equipment without the need to wait for it to cool down between disinfections.

The facemask is inserted in the equipment and held in its position by the 4 mounts (5) that allow different facemask from different manufacturers with different dimensions to be held properly. The equipment also has a transparent door (6) that allows the user to observe the inside of the equipment to ensure the proper functionality of the UV-C LED lights (1 and 2), the correct placing of the facemasks on the mounts (5), while blocking the UV-C light to avoid radiation outside the equipment and thus protecting the user.

The equipment is turned on and off with a switch (7), allowing the user to decide the time of the UV-C LED lights (1 and 2) to be active.

As appreciated in FIGS. 7,8, and 9 the equipment models are scalable, allowing the manufacturing of the equipment to disinfect 1, 4, 6 and more facemasks simultaneously with the same processes and elements described herein.

Having properly and extensively described the invention, it shall be considered sufficiently innovative and with substantial improvement from other facemask disinfectors in existence, therefore, I claim as my exclusive property the following:

1. A peculiar facemask disinfection equipment that radiates UV-C light, consisting of 4 upper UV-C LED lights and 2 bottom UV-C LED lights strategically placed for the proper UV-C light radiation targeted to the facemask; an upper cooling system and a bottom cooling system; 4 mounts for facemask placing; one transparent door and an on and off switch.

2. The disinfecting equipment described in claim 1 can be manufactured either with plastic or stainless steel and can be manufactured for 1, 4 o 6 units per equipment.

3. The disinfecting equipment described in claim 1 allows its use for long and uninterrupted periods of time without overheating nor the need for a cooling period, as well as having a switch that immediately turn on and off the equipment.
